(19) United States
(12) Patent Application Publication
(10) Pub. No.: US 2016/0183977 A1
(43) Pub. Date: Jun. 30, 2016

(54) DEVICE FOR PLACEMENT OF AN INTRAUTERINE BALLOON

(71) Applicant: THE CHARLOTTE-MECKLENBURG HOSPITAL AUTHORITY D/B/A CAROLINAS HEALTHCARE SYSTEM, Charlotte, NC (US)

(72) Inventor: Paul B. MARSHBURN, Davidson, NC (US)

(21) Appl. No.: 14/911,422

(22) PCT Filed: Aug. 19, 2014
(86) PCT No.: PCT/US2014/051613

§ 371 Date: Feb. 10, 2016

Related U.S. Application Data
(60) Provisional application No. 61/867,757, filed on Aug. 20, 2013.

(51) Int. Cl. A61B 17/42; A61B 17/12; A61B 17/02

(52) U.S. Cl. CPC A61B 17/4241; A61B 17/02; A61B 17/12136; A61B 2017/1205; A61B 2017/4216

(57) ABSTRACT

A device, system, and method that may help reduce complications associated with placing an intrauterine balloon in the uterine cavity of a patient is provided. In one aspect, the system includes an inflatable balloon, an inflation line having a distal end in communication with the balloon; an inner member comprising an elongate tubular body having a distal and proximal end; a slot extending from the distal to proximal end of the inner member, and an outer sheath in which the inner member is removably insertable. The outer sheath is configured to receive the balloon in a furled state therein such that the balloon may be introduced into the uterine cavity by transvaginally inserting the outer sheath through an endocervical canal of the patient and then introducing the balloon into the uterine cavity by moving the inner member through the outer sheath in the direction of uterine cavity.

### FIELD

The present invention relates generally to devices and methods for delivering a medical instrument transvaginally thought the cervix and into the uterine cavity, and more particularly, to a device, system, and method for placement of an intrauterine balloon into the uterine cavity.

### BACKGROUND

A wide variety of medical procedures exist that require transvaginal entry into the uterine cavity. For example, in hysteroscopy a hysteroscope is inserted through the vagina and into the cervical opening after cervical dilation. Hysteroscopy allows for the diagnosis of intrauterine pathology and may also be used as a method for surgical intervention, for example, hysteroscopic surgery.

Hysteroscopic surgery is frequently performed to remove benign tumors of the uterus, such as polyps and myomas, incision of a fibrous septum, and to breakdown intrauterine scar tissue. Generally, hysteroscopic surgery is performed with a surgical instrument that is passed through a surgical port of the hysteroscope, and then into the uterine cavity. Such a surgical instrument may include an energized wire, knife, radiofrequency device, or the like, that is used to carry out the surgical procedure.

Following the surgical operation, there is a concern that bridging adhesions may form in which the walls of the endometrial cavity stick to each other and form connective scar tissue. Such bridging adhesions may prevent pregnancy, cause miscarriage, produces complications during pregnancy, and be related to menstrual disorders. To prevent the formation of such bridging adhesions, intrauterine spacer balloons have been developed to prevent the adhesion of the endometrial cavity walls to each other. In this regard, FIG. 1 illustrates a device 10 for introducing an intrauterine spacer balloon 12 into the uterine cavity of the patient. The device generally includes the intrauterine spacer balloon 12, a syringe 14 for introducing a fluid into the balloon, and an inflation line 16 (e.g., a flexible tube or hose) that extends between a distal end of the syringe and the balloon. The end of the syringe may be attached to the inflation line via a flexible end-piece that makes a fluid tight connection, and is configured to be removably attached to the syringe.

The standard method of placement of the spacer balloon is to roll the uninflated balloon so that it may be grasped lengthwise with forceps to insert the balloon transvaginally through the endocervical canal and into the uterine cavity. Once the balloon is positioned in a desired location, the forceps must be withdrawn without displacing the spacer balloon from the uterine cavity. Thereafter, the balloon is inflated and detached from the syringe. The inflated balloon typically remains in the uterine cavity for about four days to allow for initial healing of the uterine cavity. The balloon is then deflated and removed. The presence of the inflated balloon, which typically has a shape mirroring the intrauterine cavity, provides a physical barrier to help prevent the walls of the endometrial cavity from sticking to each other. FIG. 2 shows the balloon 12 in an inflated state within the uterine cavity 20 of a patient.

Unfortunately, positioning of the intrauterine spacer balloon is difficult to place following surgery. The spacer balloon, while being flexible, is unwieldy to roll and difficult to grasp with forceps without becoming unrolled. In particular, after grasping with the forceps, the edges of the rolled up balloon tend to “flare out” making it difficult to insert the baboon through the endocerival canal. To overcome this difficulty, may be necessary to use a tenaculum, such as uterine or cervical tenactum to grasp the cervix and thereby provide counter pressure to help force the balloon through the endocerival canal. In some cases, this may result in the tenaculum becoming dislodged due to the amount of the force being applied. This can result in lacerations to the cervix, which may cause bleeding and frequently requires surgical repair, and thus, causing patient injury, blood loss, and increased operating room time. As a result of these difficulties, many surgeons may decline or abort the use the spacer balloon and thereby deny the patient the benefits the balloon provides.

Accordingly, there exists a need for a device, system, and method that improves the ability and safety of inserting medical devices transvaginally through the cervix and into the uterine cavity.

### SUMMARY

Embodiments of the present invention are directed to a device, system, end method that may help reduce complications associated with placing an intrauterine balloon in the uterine cavity of a patient. In particular, embodiments of the present invention should help improve the successful rate of intrauterine balloon placement and thereby help reduce complications associated with cervical laceration and over dilation.

In one embodiment, the invention provides a device for transvaginally inserting a medical device into the uterine cavity of a patient in which the device comprises an inner member having an elongate tubular body having a distal and proximal end, a slot extending from the distal to proximal end of the inner member; and an outer sheath having a longitudinally extending body in which the inner member is removably insertable.

In one aspect, the device is configured to be used for the intrauterine balloon placement of intrauterine spacer balloons. In particular, the outer sheath defines a cannula having a tubular shaped body that is configured to receive a non-inflated spacer balloon (e.g., in a furled state) therein. The slot of the inner member is configured to receive an inflation line of the spacer balloon assembly therein. The inner member can then be inserted into the outer sleeve so that its distal end abuts against the uninflated balloon, which is positioned in the outer sleeve.

After assembly, the assembled system is transvaginally inserted through endocervical canal of the patient. In one embodiment the system is advanced through the endocervical canal until the distal end of the outer sleeve is positioned adjacent to the uterine cavity. Preferably, the distal end of the outer sleeve is advanced until it is adjacent to the internal os of the patient's cervix. Once the device (e.g. outer sleeve) is properly positioned, the inner member and balloon assembly are collectively advanced through the outer sleeve in the direction of the distal end of the outer sleeve and towards the uterine cavity.

As the inner member is advanced, the distal end thereof pushes the furled balloon forward so that balloon is introduced into the uterine cavity of the patient. In one embodiment, the inner member may function similar to that of a plunger to drive the balloon forward through the outer sleeve. Further advancement of the inner member advances the balloon out of the outer sleeve and into the uterine cavity. At this point, the balloon can then be expanded by introducing a fluid via a syringe that is connected to the inflation line.

A further aspect of the invention provides a system for transvaginally introducing an intrauterine balloon into a uterine cavity of a patient in which the system comprises an inflatable balloon; an inflation line having a distal end in communication with the balloon; an inner member comprising an elongate tubular body having a distal and proximal end; a slot extending from the distal to proximal end of the inner member; and an outer sheath in which the inner member is removably insertable. The outer sheath is configured to receive the balloon in a furled state therein such that the balloon may be introduced into the uterine cavity by transvaginally inserting the outer sheath through an endocervical canal of the patient and then introducing the balloon into the uterine cavity by moving the inner member through the outer sheath in the direction of uterine cavity.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 shows a conventional system for placement of an intrauterine spacer balloon in a patient's uterine cavity;

FIG. 2 shows an intrauterine spacer balloon in an inflated state in the uterine cavity of a patient;

FIG. 3 shows an exploded view of a device in accordance with at least one embodiment of the invention for introducing a medical instrument into the uterine cavity of a patient;

FIG. 4 shows the device of FIG. 3 in which the inner member is partially positioned in the outer sleeve;

FIG. 5 shows a system for implanting a medical device Into the uterine cavity in which the system is in a partially assembled state.

FIGS. 6A-6B and 7A-7B shown an exemplary process of assembling a device in accordance with at least one embodiment of the invention;

FIG. 8 shows an embodiment of the invention in accordance with at least one aspect of the invention; and

FIGS. 9A-9D show a process of utilizing the device of FIG. 1 for placement of an intrauterine balloon in a patient.

### DETAILED DESCRIPTION

The present invention now will be described more fully hereinafter with reference to the accompanying drawings, in which some, but not all embodiments of the Invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

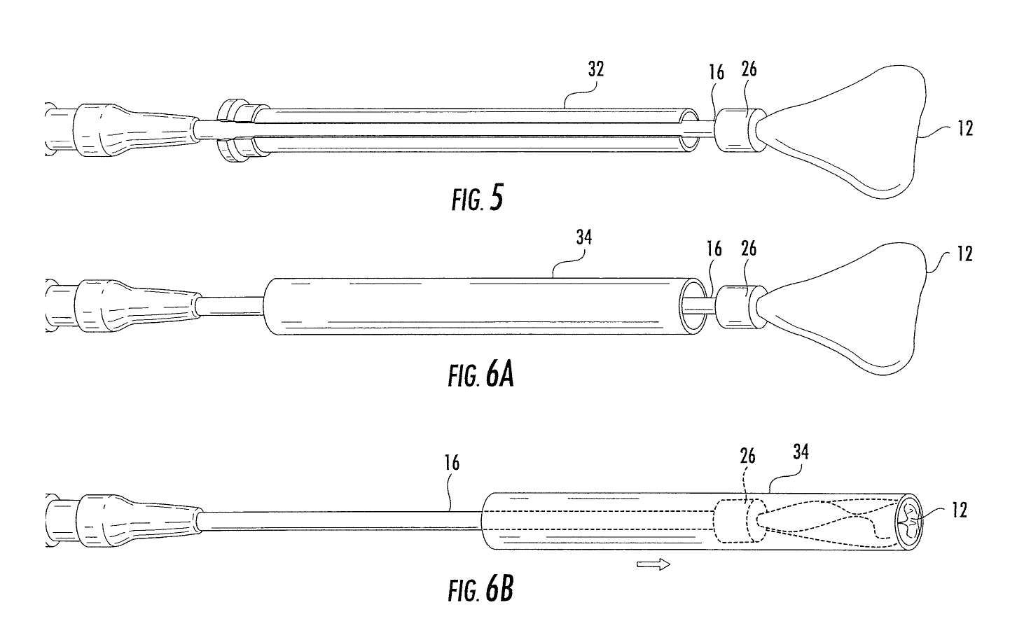

In a first aspect, embodiments of the present invention are directed to a device and system for placement of an intrauterine spacer balloon in a patient. With reference to FIG. 3, a device for introducing a medical instrument into the uterine cavity of a patient is illustrated and broadly designated by reference number 30. The device 30 includes an inner member 32 having a tubular shaped elongate body, and an outer sleeve 34 in which the inner member 32 is removably insertable. The device 30 including the inner member 32 and outer sleeve are collectively configured to be transvaginally inserted through the endocervical canal so that a distal end of the device is positioned above the restrictive barrier of the endocervix, towards the uterine cavity of the patient.

The inner member 32 includes a distal end 36, a proximal end 38, and a longitudinally extending inner channel 40 that extends between the proximal end 38 and the distal end 36 of the inner member 32. A longitudinal slot 42 extends between the proximal end 38 and distal end 36 of the inner member and provides communication between inner channel 40 and an outer surface 44 of the inner member. In one embodiment, the inner member comprises a slotted cannula defining an inner plunger of device 30.

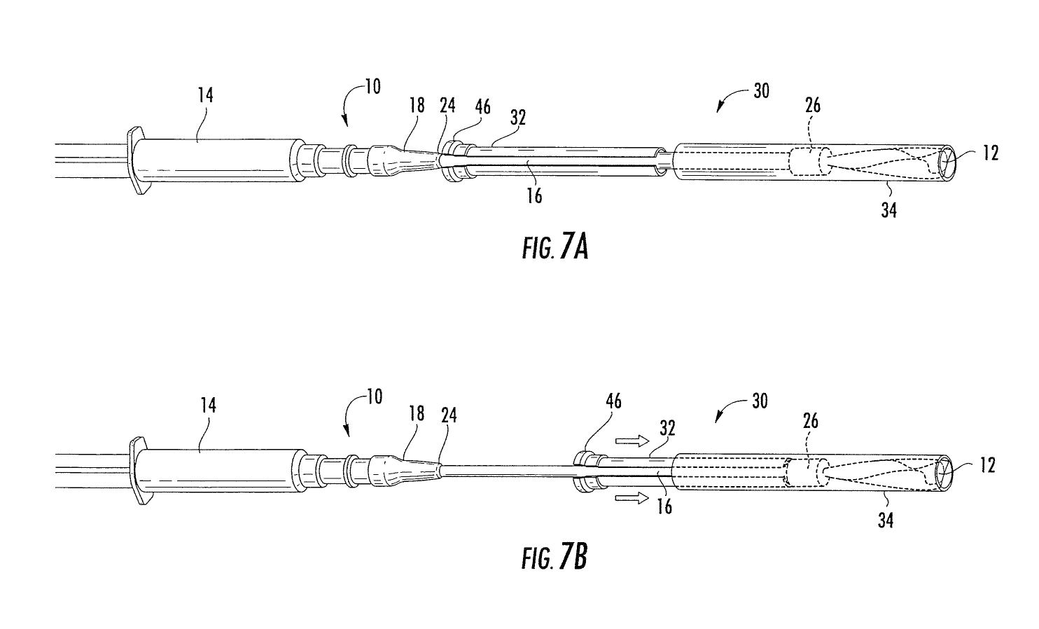

The outer sleeve 34 generally comprises a tubular shaped body that extends longitudinally between a proximal end 50 and a distal end 52. As noted previously, the inner member 32 is sized so that it may be removably inserted into the outer sleeve 34. In this regard, FIG. 4 shows the device 30 with the inner member partially inserted into the outer sleeve.

The inner member and outer sleeve may comprise any material suitable for medical applications, such as a polymeric material. Suitable materials for the inner member and outer sleeve may include a wide variety of polymers including silicone rubber, nitinol, nylons, polyurethanes, polyolefins, such as polyethylenes and polypropylenes, polyesters, such as polyethylene terephthalate (PET) and polyethylene terephthalate (PETE) latex, polycarbonates, polysulfones, polytetrafluoroethylene (TEFLON®), acrylics, polyetherketones, hydrophilic polymers, and thermoplastic elastomers. In one particular embodiment, the inner member and outer sleeve may comprise a flexible silicone material. In one embodiment, the inner member and outer sleeve may comprise a flexible material. In other embodiment, one or more of the inner member and outer sleeve may comprise a material that is rigid or semi-rigid. In a preferred embodiment, the outer sleeve is rigid or semi-rigid, and the inner member comprises a flexible material.

In one embodiment, the outer sleeve may have an outer diameter ranging from about 0.5 to 1 cm, and in particular, from about 0.7 to 0.85 cm. In a preferred embodiment, the outer diameter of the outer diameter is from about 0.75 to 0.85 cm. The length of the device 30 (e.g., the outer sleeve and the inner member may range from about 5 to 25 cm, and most preferably has a length 10 to 20 cm, with a length of about 15 cm being particularly preferred.

In some embodiments, the inner member may have a length that is longer than that of the outer sleeve. For example, the length of the inner elongate member may be from about 0.5 to 5 cm longer than that of the outer sleeve, and is preferably from about 1 to 3 cm longer than the length of the outer sleeve.

In a preferred embodiment, the proximal end 40 of the inner member 32 includes a collar 46 having an outer diameter that is larger than an inner diameter of the outer sleeve so as to prevent the inner elongate member from being completely inserted into the outer sleeve. In some embodiments, the inner member may include a handle or gripping portion disposed towards the proximal end to help assist in the insertion and removal of the inner member into and out of the outer sleeve.

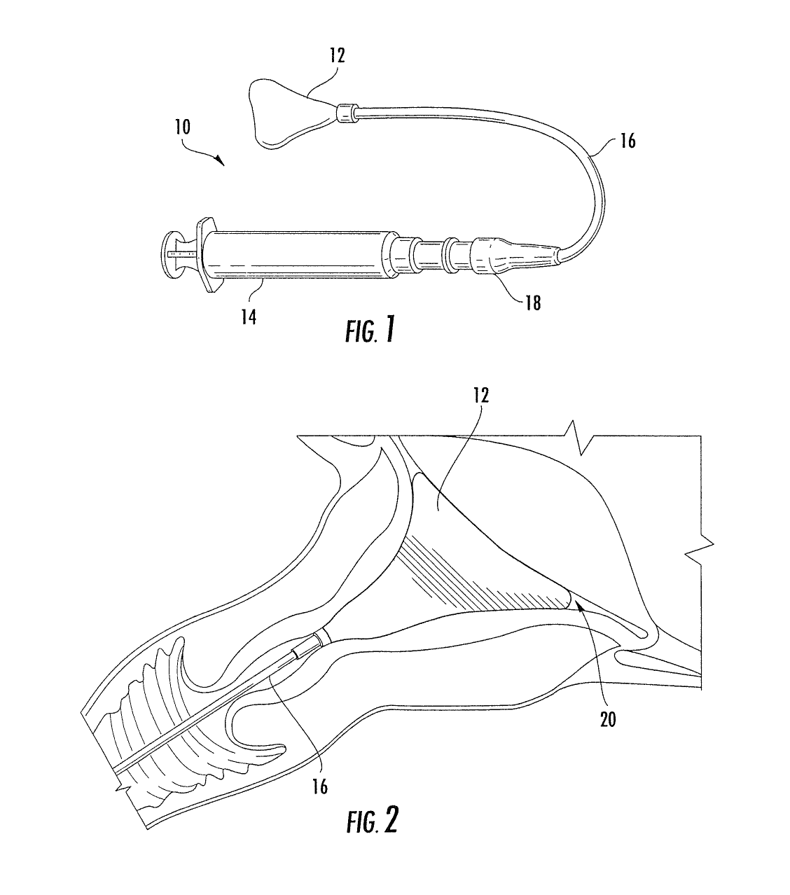

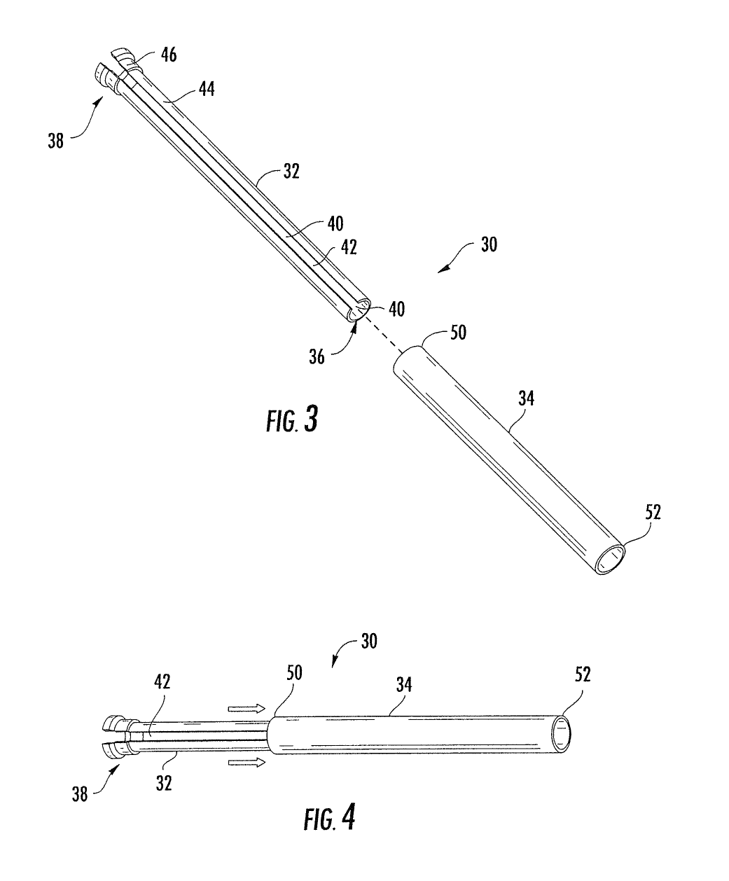

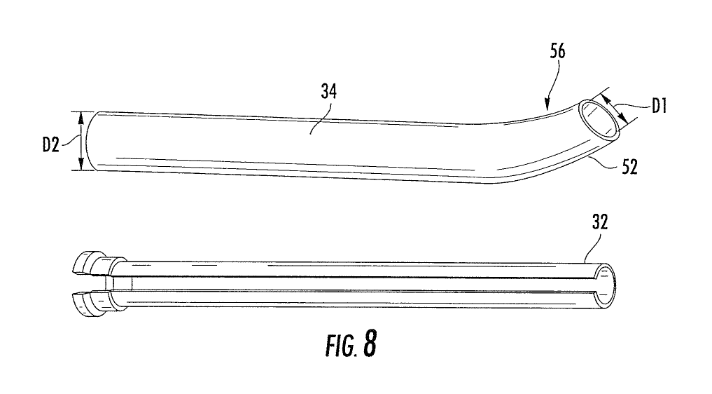

In one aspect, the invention may include a device 30 having an inner member 32 as described above, a cooperating outer sheath 34, and a balloon stent assembly (see FIG. 1, for example). In this regard, FIG. 5 shows a system for implanting a medical device into the uterine cavity of a patient in which the system is in a partially assembled state. More specifically, FIG. 5 shows a balloon stent assembly 10 having an inflation line 16 disposed within the inner channel of the inner member 32.

The balloon stent assembly typically includes an intrauterine balloon 12, a syringe 14, and a flexible inflation line 16 extending between the syringe and the balloon: The inflation line provides fluid communication between the syringe and the balloon. Generally, the proximal end of the inflation line includes a connector 18 for connecting the inflation line to the base of the syringe. For example, the connector 18 may include a ball valve that allows introduction of a fluid into the balloon, but prevents reflux of the fluid back out of the connector. In some embodiments, the balloon stent assembly may include a collar or neck 26 at the base of the balloon 12. The balloon stent assembly may be a conventional balloon uterine stent, such as available from COOK® MEDICAL.

The intrauterine balloon typically comprises a biocompatible elastomeric material. In one embodiment, the intrauterine balloon comprises a silicone rubber, e.g., SILASTIC® Q7-4850 available through Dow Coming. The intrauterine balloon 12 preferably has a shape that accommodates the shape of the uterine cavity. For example, in some embodiments the intrauterine balloon may have a slightly conical shape with outer surfaces configured for engaging the walls of the uterine cavity. In one embodiment, the diameter of the balloon may range from about 2 to 5 cm, and in particular from about 3 to 4 cm.

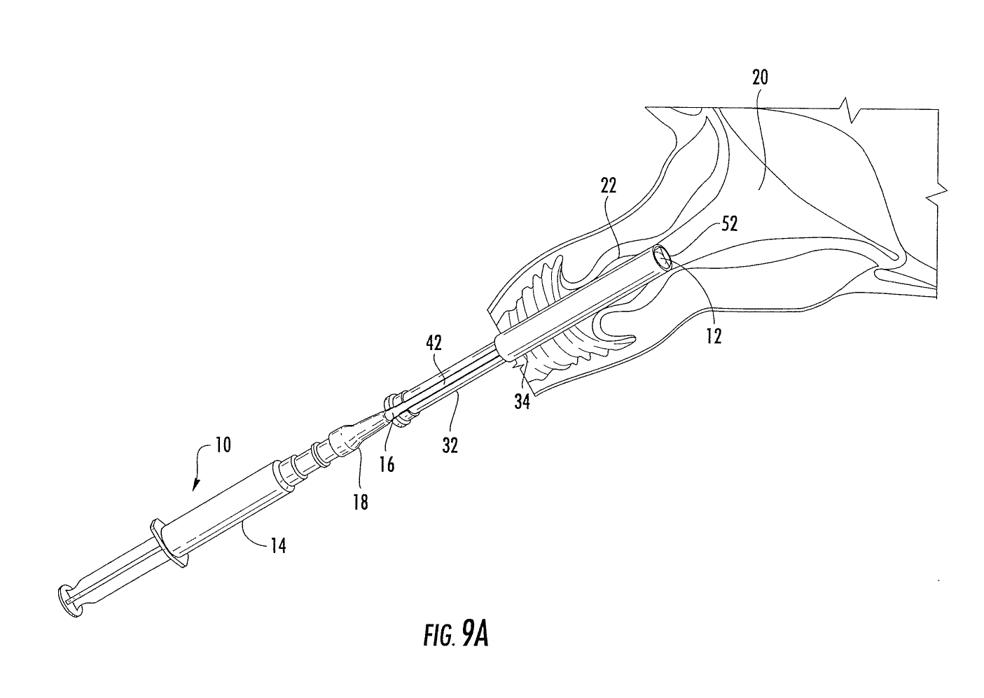

FIGS. 6A-6B and 7A-76 collectively illustrate an example of a process of assembling a system for introducing a medical device, such as an intrauterine balloon, utilizing device 30. As shown in FIG. 6A, the system may be assembled by inserting the balloon stent assembly through the outer sleeve 34. Preferably, the inflation line extends through the outer sleeve so that the collar 26 and balloon 12 are positioned adjacent to the distal end 52 of the outer sleeve.

In a subsequent step as can best be seen in FIG. 6B, the balloon 12 is in a furled state (e.g. rolled or folded) and positioned within the outer sleeve. In some embodiments, the balloon may be positioned within the outer sleeve towards the distal end of the outer sleeve. Preferably, the balloon is retracted completely into the outer sleeve 34 in a compacted state so there are no flared edges. Furling of the balloon may be performed by folding or rolling up the edges of the balloon followed by slidingly inserting the collar and balloon into the distal end of the outer sleeve.

In the illustrated embodiment of FIGS. 5, 6A and 68, the inflation line 16 includes a connector 18 for attachment to a syringe. Generally, the connector 18 has a diameter that permits the connector to pass through the outer sleeve 34. Typically, the connector 18 is configured to be releasably attached to the base of the syringe. In one embodiment, the connector comprises a flexible cone that creates a fluid tight connection with the inflation line and syringe. As noted previously, the connector may include an associated internal valve, such as a ball valve, that allows introduction of a fluid into the balloon, but prevents reflux of the fluid back out of the connector.

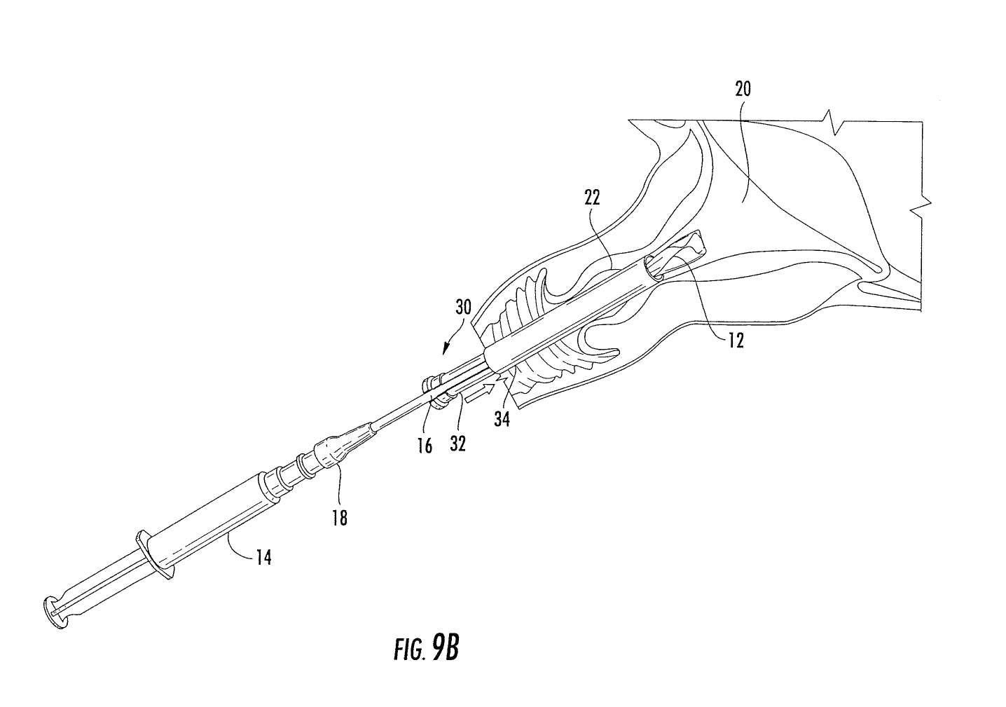

In a next step, assembly of the system may be completed by positioning the inflation line 16 through the slot 42, and into the inner channel 40 of the inner member 32, and then sliding the inner member 32 into the outer sleeve 34. In this regard, FIGS. 7A-7B show the steps of completing the assembly of the system. In FIG. 7A, the inflation line 16 is inserted into the inner channel 40 of the inner member via longitudinal slot 42. Thereafter, the inner member 32 is slidingly inserted into outer sleeve 34. Preferably, the inner member 32 is inserted into the outer sleeve 34 until the distal end 36 contacts the collar 26 of the balloon stent assembly.

As explained in greater detail below, the distal end of the inner member may be sized so that it is about the same or slightly smaller than the diameter of collar 26. In this way, the inner member can be positioned up against the collar so that it can be used to drive the collar and balloon out of the distal end of the outer sleeve. Preferably, the inner member 32 is adjacent to or abuts. the collar 26 at the distal end of the inner member 32. In this manner, forward movement of the inner member 32 into and through the outer sleeve 34 pushes the balloon in the direction of the distal end of the outer sleeve so that the balloon may then be introduced into the uterine cavity while the position of the outer sleeve remains substantially stationary relative to the endocervical canal of the patient. In this way, the inner member 32 functions similar to a plunger to introduce the balloon into the uterine cavity.

It should be recognized that the system may be assembled in alternative or modified techniques to that described above. For example, in some aspects the system may be assembled by first inserting the intrauterine balloon 12 and inflation line 16 into the longitudinal slot 42 of the inner member 32. In this embodiment, the balloon 12 is typically in a furled (e.g. rolled or folded) so that it is substantially disposed within the longitudinal slot 42 of the inner member. As previously mentioned, it is preferred that the balloon is in a compacted state and includes no flared edges. Furling of the balloon may be performed by folding or rolling up the edges of the balloon followed by slidingly inserting the balloon into the distal end of the longitudinal slot 42. Thereafter, the inner member having the furled balloon may be inserted into the outer sheath.

Although FIGS. 5 and 6 show the connector as not be connected to the syringe, whereas FIGS. 7A-7B show the syringe connected, it should be recognized that the exact timing for attachment of the inflation line to the syringe is not critical and may occur prior to inserting the inner member into the outer sleeve, or after the device 30 has been introduced into the endocervical canal of the patient.

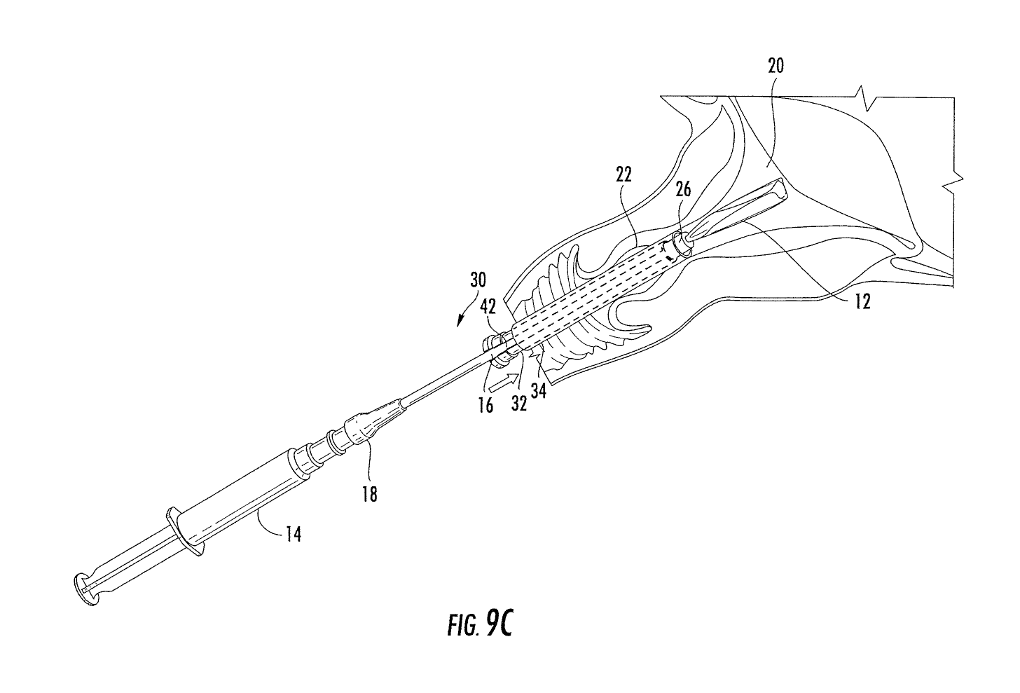

With reference to FIG. 8, an embodiment of the device 30 is shown in which the distal end 52 of the outer sleeve 34 includes a slightly curved region 56 to assist in inserting the device 30 into the endocervical canal. In this aspect, the inner member 32 may comprise a flexible material that it is capable of banding and conforming to the shape of the outer sleeve.

In some aspects, the distal end of the outer sleeve may also be slightly tapered to assist in the insertion of the outer sleeve through the patient's cervical canal. In this regard, FIG. 8 also shows an embodiment in which the outer sleeve includes a distal end 52 having a diameter D2 that is slightly smaller than the diameter D1 at the proximal end of the outer sleeve. The slightly curved and tapered region may help allow the distal end of the outer sleeve to accommodate the bioelastic nature of the cervix, much in the way in which curved and tapered dilators have been used to progressively open the cervix so as to help prevent tissue injury and bleeding.

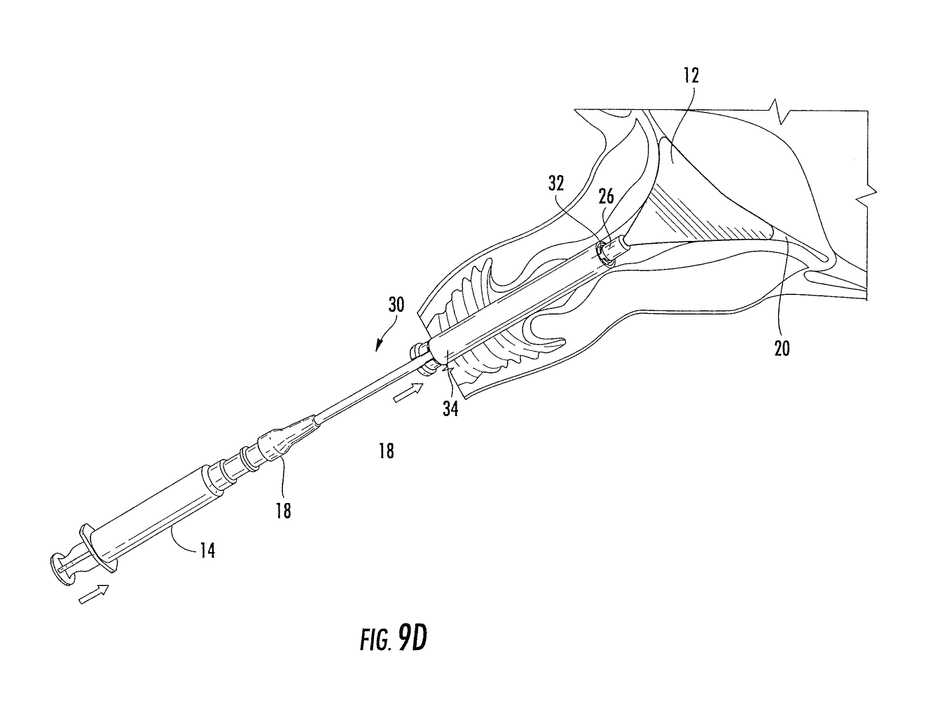

FIGS. 9A-9D Illustrate an exemplary process of deploying an intrauterine balloon in the uterine cavity of a patient. In FIG. 9A, the assembled system (e.g., device 30 and balloon sent assembly 10) are collectively transvaginally inserted through endocervical cane 22 of the patient. In one embodiment, the assembled device 30 is advanced through the endocervical canal until the distal end 52 is positioned adjacent to the uterine cavity 20. Preferably, the distal end of the outer sleeve is advanced until it is adjacent to the internal os of the patient's cervix.

Once the device (e.g. outer sleeve) is properly positioned, the inner member 32 and balloon assembly are collectively advanced through the outer sleeve 34 in the direction of the distal end 52 thereof (e.g., towards the uterine cavity 20). As can best be seen in FIG. 9B, the inner member 32 is advanced through the outer sleeve 34 towards the direction of the distal end of the outer sleeve. As the inner member is advanced, the distal end thereof pushes the furled balloon forward so that balloon is introduced into the uterine cavity of the patient. As noted previously, the inner member 32 may function similar to that of a plunger to drive the balloon forward through the outer sleeve 34. In one embodiment, the distal end 36 of the inner member is configured to push against the collar 26 of the balloon stent assembly, and thereby collectively drive the collar and balloon through the outer sleeve.

As shown in FIG. 9C, further advancement of the inner member advances the balloon out of the outer sleeve 34 and into the uterine cavity 20. At this point, the balloon can then be expanded by introducing a fluid via the syringe 14.

FIG. 9D shows the balloon 12 in an expanded state within the uterine cavity 20 of the patient. Following inflation of the balloon, the inflation line 16 can be disconnected from the syringe, and the device 30 (e.g., inner member and outer sleeve) can be withdrawn from the cervix.

After inflation, the inflated balloon typically remains stationary within the uterine cavity. The inflation line may then be brought through the slot 42 of the inner member 32, and then the inner member is withdrawn from the outer sleeve 34. Thereafter, the outer sleeve may be withdrawn from the cervix over inflation line 16. The inflation line is then typically folded and placed into the vagina so that it does not protrude onto the perineum. In some embodiments, the above placement may be assisted by visualization via transabdominal ultrasound.

From the foregoing discussion, it can be seen that embodiments of the present invention may help reduce complications associated with placing an intrauterine balloon in the uterine cavity. In particular, embodiments of the present invention should help improve the successful rate of intrauterine balloon placement and thereby help reduce complications associated with cervical laceration and over dilation.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A device for transvaginally inserting a medical device into the uterine cavity of a patient, the device comprising:
an inner member comprising an elongate tubular body having a distal and proximal end;
a slot extending from the distal to proximal end of the inner member; and
an outer sheath having a longitudinally extending body in which the inner member is removably insertable, wherein the device is configured to be transvaginally inserted through a endocervical canal of the patient.

2. The device of claim 1, wherein the proximal end of the inner member includes a collar having a diameter larger than that of an inner diameter of the outer sheath.

3. The device of claim 1, wherein the outer sheath is from about 10 to 20 cm in length.

4. The device of claim 1, wherein the inner member is from about 0.5 to 5 cm longer than the length of the outer sheath.

5. The device of claim 1, wherein the inner member and the outer sheath comprises a polymeric material.

6. The device of claim 1, wherein the distal end of the outer sheath has a curved shape, and wherein the inner member is tubular with open distal and proximal ends, each having a circular perimeter, and wherein the distal and proximal ends are both free ends that are detached from other components.

7. The device of claim 1, wherein the outer sheath comprises a rigid or semi-rigid material and the inner member comprises a flexible material.

8. A system for transvaginally introducing an intrauterine balloon into a uterine cavity of a patient, the system comprising:
an inflatable balloon;
an inflation line having a distal end in communication with said balloon;
an inner member comprising an elongate tubular body having a distal and proximal end;
a slot extending from the distal to proximal end of the inner member; and
an outer sheath in which the inner member is removably insertable, said outer sheath configured to receive the balloon in a furled state therein,
wherein the balloon is introduced into the uterine cavity by transvaginally inserting the outer sheath through an endocervical canal of the patient and then introducing the balloon into the uterine cavity by moving the inner member through the outer sheath in the direction of uterine cavity.

9. The system of claim 8, further comprising a syringe in fluid communication with a proximal end of the inflation line.

10. The system of claim 8, wherein the inflation line is releasably attached to the syringe.

11. The system of claim 8, wherein the proximal end of the inner member includes a collar having a diameter larger than that of an inner diameter of the outer sheath.

12. The system of claim 8, wherein a base of the balloon includes a collar having a diameter that is about the same size or larger as a diameter of the distal end of the inner member.

13. The system of claim 8, wherein the distal end of the outer sheath includes a region that is tapered and has a curve shape.

14. The system of claim 13, wherein the outer sheath comprises a rigid or semi-rigid material and the inner member comprises a flexible material.

15. A method for transvaginally introducing an intrauterine balloon into a uterine cavity of a patient, the method comprising:
positioning an inflatable balloon in a furled state in an outer sheath;
inserting an inner member comprising an elongate tubular body having a proximal end, a distal end, and a longitudinal slot extending therebetween into the outer sheath
transvaginally inserting the outer sheath through an endocervical canal of the patient, the outer sheath having a longitudinal, tubular shaped body;
advancing the inner member through the outer sheath until the balloon is moved out of the outer sheath and into the uterine cavity; and
introducing a fluid into the balloon to expand the balloon in the uterine cavity.

16. The method of claim 15, wherein the step of introducing a fluid into the balloon comprises moving the fluid from a syringe through an inflation line that is in communication with said balloon.

17. The method of claim 16, further comprising the step of disconnecting the syringe from the balloon after the balloon has been expanded.

18. The method of claim 15, further comprising the step of withdrawing the inner member and outer sheath from the patient after the balloon has been expanded.

19. The method of claim 15, wherein the step of inserting an inner member further comprises advancing the inner member through outer sheath until the distal end of the inner member is adjacent to the balloon.

20. The device of claim 1, further comprising an intrauterine balloon and an inflation line attached to the intrauterine balloon, wherein the intrauterine balloon is held in a deflated, furled shape inside the outer sheath with the inflation line extending in the slot of the inner member and out of a proximal end of the outer sheath, and wherein the inner member slidably advances in the outer sheath to push the balloon out of the outer sheath allowing the balloon to increase in size in position in a uterus of a patient.
